(19) United States
(12) Patent Application Publication
(10) Pub. No.: US 2024/0301466 A1
(43) Pub. Date: Sep. 12, 2024

(54) EFFICIENT DUPLEX SEQUENCING USING HIGH FIDELITY NEXT GENERATION SEQUENCING READS

(71) Applicant: University of Washington, Seattle, WA (US)

(72) Inventor: Stephen J. SALIPANTE, Seattle, WA (US)

(73) Assignee: University of Washington, Seattle, WA (US)

(21) Appl. No.: 18/578,523

(22) PCT Filed: Jul. 13, 2022
(86) PCT No.: PCT/US2022/036951

§ 371 Date: Jan. 11, 2024

Related U.S. Application Data
(60) Provisional application No. 63/222,340, filed on Jul. 15, 2021.

(51) Int. Cl. C12Q 1/6806; C12N 15/10; C12Q 1/6869

(52) U.S. Cl. CPC C12Q 1/6806; C12N 15/1065; C12Q 1/6869

(57) ABSTRACT

Embodiments of the present disclosure provide a method for detecting one or more genetic variants in a biological sample. Embodiments of the method include preparing an error-corrected nucleic library for sequencing, wherein the nucleic acid library comprises a double stranded nucleic molecule comprising a hairpin adapter, wherein the hairpin adapter covalently joins each strand of the double stranded nucleic molecule into a single covalently linked duplex strand for self-correction of sequencing errors.

### CROSS-REFERENCE(S) TO RELATED APPLICATION(S)

This application claims the benefit of U.S. Provisional Application No. 63/222,340, filed Jul. 15, 2021.

### BACKGROUND

Next-generation DNA sequencing (NGS) has been embraced by the clinical oncology community, where its ability to scalably examine hundreds to thousands of targets now routinely enables identification of prognostic and therapeutically actionable markers supporting the practice of precision medicine. Nevertheless, standard implementations of existing sequencing technologies (whole genome or targeted gene sequencing), are limited in application by a relatively high error rate, leading to poor sensitivity for detecting low-prevalence mutations. Various methods have been proposed to bypass this issue by error correcting NGS sequence reads, but all have practical shortcomings. Notably, prevalent techniques in the field (such as “Duplex Sequencing”) require initial labeling of library fragments with a degenerate “unique molecular identifier” (UMID) DNA sequence, then redundantly sequencing each fragment at high depth to produce an error-corrected consensus. UMID-mediated approaches incur excessive read depth requirements and accordingly high sequencing costs, limiting the number of gene targets that can be examined in a single assay. As a practical outcome, error-corrected sequencing has seen little uptake in the clinical diagnosis and characterization of malignancy.

Accordingly, there remains a need for fast, efficient, facile, inexpensive, and accurate sequencing approaches that leverage the power of NGS platforms. To be useful, e.g., in the clinical practice of precision medicine, sequence read error correction strategies must exhibit multiple properties that are incompletely addressed by existing paradigms: (1) scalability—the approach can interrogate large numbers of genomic targets (i.e., from a few genes to the entire exome or genome); (2) cost-effectiveness—the total cost from specimen to result must be inexpensive enough for routine use; (3) ease of use—the approach must be compatible with clinical workflows and clinical testing volumes; (4) efficiency—the approach requires a minimal number of sequencing reads for compatibility with low-to-mid throughput sequencing platforms available to most clinical laboratories; (5) ultrasensitivity—detection of low-prevalence mutant alleles in a very large background of unaltered genes (<1 in 10,000 mutant alleles); and (6) quantitative precision—the true frequency of variants can be accurately determined. The present disclosure addresses these and related needs.

### SUMMARY

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

In accordance with the foregoing, in one aspect of the invention, the disclosure provides a method to prepare an error-corrected nucleic acid library for sequencing, the method comprising: providing a double stranded nucleic acid molecule, comprising a positive strand and a negative strand, wherein the positive strand and the negative strand are substantially complementary, and wherein the double stranded nucleic acid molecule has a first end and a second end; covalently attaching a first sequencing adapter to the positive strand at the first end of the double stranded nucleic acid molecule; covalently attaching a second sequencing adapter to the negative strand at the first end of the double stranded nucleic acid molecule; and covalently attaching a first end of a single hairpin adapter to the positive strand at the second end of the double stranded nucleic acid molecule and covalently attaching a second end of the single hairpin adapter to the negative strand at the second end of the double stranded nucleic acid molecule, wherein the single hairpin adapter covalently joins the positive strand and the negative strand into a single covalently linked duplex strand for self-correction of sequencing errors.

In another aspect of the invention, the disclosure provides for a linked duplex nucleic acid molecule produced by the method described above.

In another aspect of the invention, the disclosure provides a method for detecting one or more genetic variants in a biological sample, the method comprising: generating a sequencing library by performing the method described above, wherein the sequencing library comprises a plurality of covalently linked duplex strands each comprising a unique UMID sequence; amplifying at least a portion of the covalently linked duplex strands to produce an amplified sequencing library comprising a plurality of copies of the covalently linked duplex strands; sequencing at least a portion of the covalently linked duplex strands to obtain at least one sequence read comprising a first subsequence corresponding to at least a portion of the positive strand of the double stranded nucleic acid molecule and a second subsequence corresponding to at least a portion of the negative strand of the double stranded nucleic acid molecule; and detecting a presence or absence of one or more genetic variants in the biological sample, by comparing the sequence of the first subsequence to the sequence of the second subsequence, wherein one or more variants observed in both subsequences are genetic variants.

In another aspect of the invention, the disclosure provides a kit comprising: a first sequencing adapter, a second sequencing adapter, a single hairpin adapter, one or more primers that hybridize to sequences in the first sequencing adapter and/or second sequencing adapter, or a complement thereof, and free nucleotides (dNTPs), a DNA polymerase, a ligase, and written indicia instructing the performance of the method described above.

In another aspect of the invention, the disclosure provides a kit comprising a first sequencing adapter, a second sequencing adapter, a single hairpin adapter, a transposome, one or more primers that hybridize to a transposon sequence, a DNA polymerase, a ligase, and written indicia instructing the performance of the method described above.

### DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIG. 1. Schematically illustrates conventional duplex sequencing vs. linked duplex sequencing.

Conventional Duplex Sequencing (A-H). (A) DNA is sheared and A-tailed. (B) ligation of Y-adapters containing i5 and i7 sequencing adapters (yellow/green) and a unique, random, double-stranded UMID (red and blue) to generate molecules labeled with two unique tags. (C) PCR copies the strands of the tagged template molecule. The two strands carry reciprocal copies of the two UMIDs. (D) Paired-end sequencing is performed to recover UMID sequences and genomic DNA. (E) Reads sharing the same set of tags (red and blue) are grouped as those originating from the same template molecule; the orientation of the tags identifies reads as coming from one of the two original strands. UMIDs with only one strand recovered, or insufficient redundant sequencing of both strands are discarded (other colors). (F and G) an error-corrected “consensus” of each strand is generated. (H) The sequences of the two strands are compared, and only variation observed in both are accepted as true mutations. Linked Duplex Sequencing (A-G). (A) DNA is sheared and A-tailed. (B) A Y-adapter containing i5 and i7 sequencing adapters is ligated to one end, and hairpin adapter integrating a UMID (blue) is ligated to the other to generate molecules labeled with a unique tags. (C) PCR converts the ligated product to a fully double stranded molecule, in which the two strands of the original template are covalently joined. (D) Paired end sequencing is performed, with each read interrogating one of the two original strands. The i7 index read is repurposed to interrogate the sequence of the UMID. (E) Every read with a unique UMID is carried forward for analysis. Reads carrying the same UMID are deduplicated to avoid representational bias. (F and G) The sequences of the two strands from each template molecule are compared, and only variation observed in both are accepted as true mutations.

FIG. 2. Graphically illustrates read depth requirements of conventional and linked duplex sequencing. Data are shown using 150 bp reads at two different target depths (7 k and 28 k) for genomic targes of varying sizes. Capacity of various Illumina instruments is in green. Note log scale on both axes.

FIG. 3. Illustrates a gel from a linked duplex library preparation. A 128 bp amplicon is used as template. A 251 bp product occurs when Y-adapters ligate on both ends. The intended, linked duplex product is observed at 450 bp after PCR.

### DETAILED DESCRIPTION

Next-generation sequencing (NGS) has rapidly become a mainstay in clinical oncology, providing large-scale genotypic information about actionable mutations that inform the practice of precision medicine. Within this space, there are many applications for which the ability to detect cancer-associated genotypes at ultra-low levels (<1 in 10,000 or more) is desirable or beneficial, including the detection and quantification of subclonal cancer-driver or drug-resistance mutations in tumors, measuring the presence of residual neoplastic cells after cancer therapy, and noninvasive approaches for early cancer detection or monitoring of recurrence. Several general technologies have been developed to interrogate variants relevant to these objectives, such as Digital Droplet PCR of specific mutations, but NGS has proven the most scalable and generalizable method of mutation detection. Consequently, NGS has been established as the method of choice to identify cancer-associated variation in most clinical applications and research laboratories alike.

Error correction methods for NGS have enabled incredible advances in the ability to identify ultra-low variation associated with human cancer, with broad potential and demonstrated clinical diagnostic applications, ranging from detecting residual malignancy cells after therapy to noninvasive oncology screening or monitoring assays, or simply improving the sensitivity of existing diagnostic assays for interpretable mutations. Despite the development of multiple, effective experimental paradigms to perform ultrasensitive error correction of NGS reads, the methods are impractical for implementation by clinical laboratories performing patient testing. This reflects deficiencies of the methods with respect to: (1) the excessive read depths required during sequencing, (2) the high costs needed to provide that sequencing, and (3) an inability to scale large numbers of genes or targets. There is thereby an unmet need for highly accurate sequencing methods that are cost-effective and allow interrogation of enough gene targets for meaningful use in clinical practice.

A barrier to detecting ultra-low variation in cancers is that variant calling by NGS is limited by a low, but measurable, error rate below which true biological variation cannot be distinguished from noise. This error rate reflects intrinsic properties of the sequencing platform and artifactual mutations induced by DNA damage during library preparation and upstream events including in vivo metabolic processes, sample fixation, and DNA extraction. The inherent error rate of the widely-used Illumina sequencing platform has been measured at ˜0.1-0.5% per base, however, the cumulative effects of these various sources of error limit the sensitivity of standard sequencing implementations to a practical limit of detection approaching ˜2-5% variant allele frequency. This cumulative error threshold severely restricts the usability and effectiveness of NGS for applications where detecting low prevalence variation is of high importance.

In response, two major strategies have emerged to achieve error correction of sequence reads so that low-frequency variants can be identified more sensitively. Each presents distinct benefits and disadvantages.

The first strategy involves computational error modeling based on the empiric observation of sequencing errors either on a general or site-specific basis. Such strategies are advantageous in that they can be applied without modifications to experimental protocols and have been shown to be effective in reducing observed error rates of sequencing to ˜0.1%, close to the theoretical error rate of NGS. Nevertheless, computational error modeling is susceptible to various batch effects that affect error rate, including sequencer cluster density, PCR conditions, and run-to-run variability. Many approaches additionally require that large numbers of samples be run in parallel or that large sets of training data be provided. Variants are called probabilistically, and performance is unpredictably dependent on the error rate of a given site and the particular variant being observed. More fundamentally, error modeling cannot identify ultra-low variation occurring below the threshold defined by the inherent error rate of sequencing itself.

The second, more effective, strategy achieves error correction by individually labeling DNA template molecules, either on the basis of randomly generated fragmentation points, or more robustly, with unique molecular identifiers (UMIDs): degenerate DNA sequence tags that distinctively label individual template molecules. During PCR amplification, this label is propagated to all copies of an original template molecule, and independent sequence reads can thus be recognized as having arisen from a common founder. Labeling enables two important capabilities: (1) quantitative accuracy of mutation detection is improved, as amplification biases can be identified and corrected; and (2) sequence error correction can be achieved by creating a consensus from reads sharing a common label, wherein true variation is recognized as being present in most members and sporadic errors are present in only a subset are dismissed.

Examples of this labeling strategy include adding UMIDs to one template strand by multiplexed PCR or molecular inversion probe capture. Such approaches reduce error rates to ˜10⁵ per base, however, artifactual mutations from amplifiable DNA lesions or errors arising during early cycles of PCR amplification cannot be distinguished from true variants, and thereby define a fixed lower limit of detection.

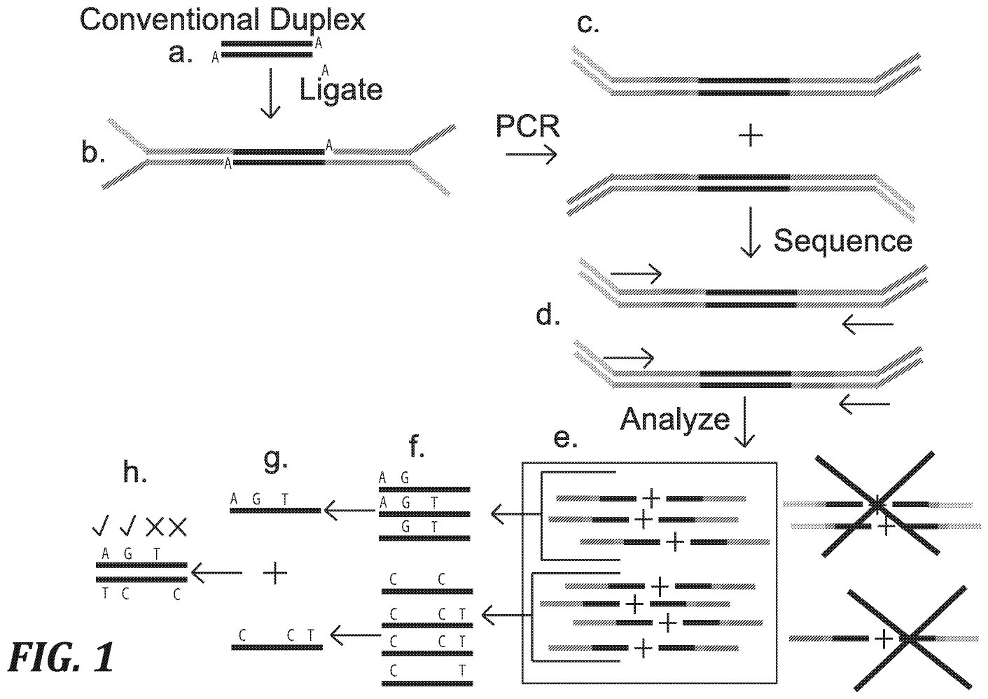

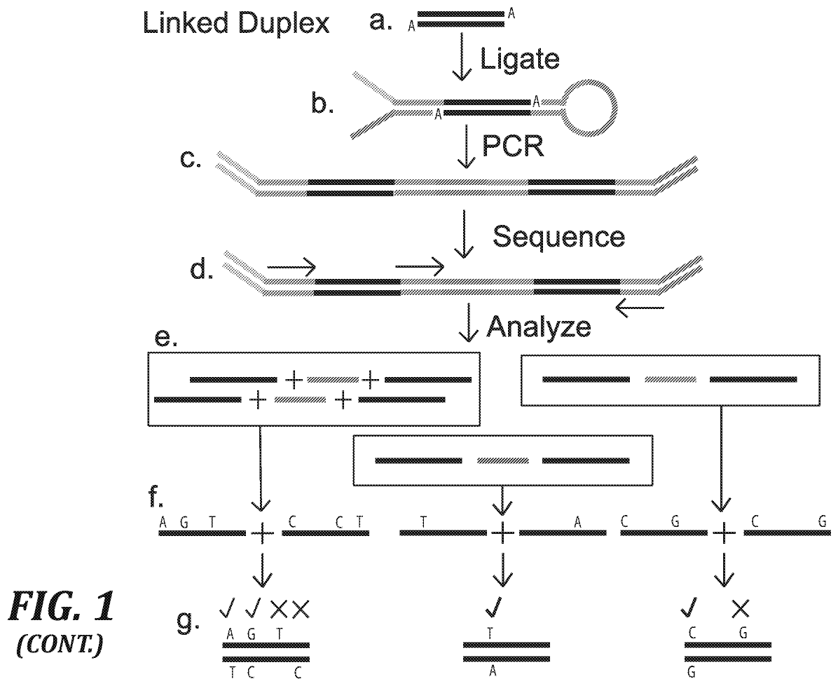

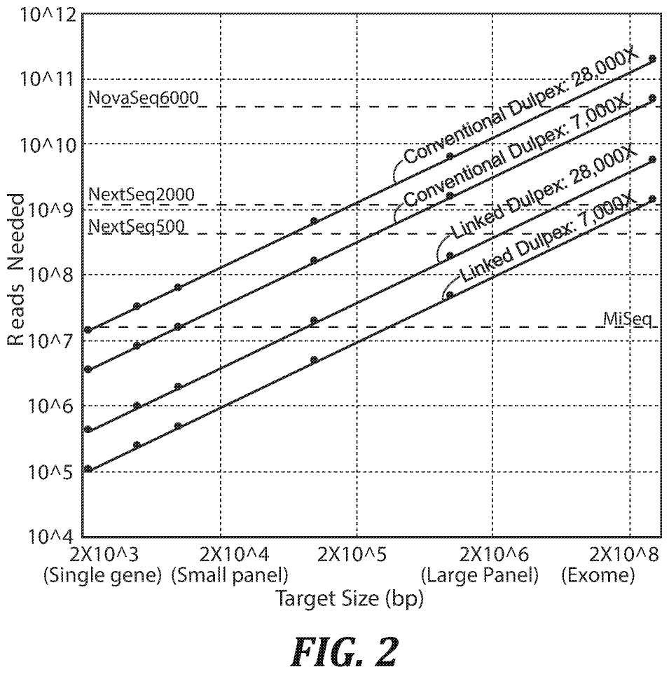

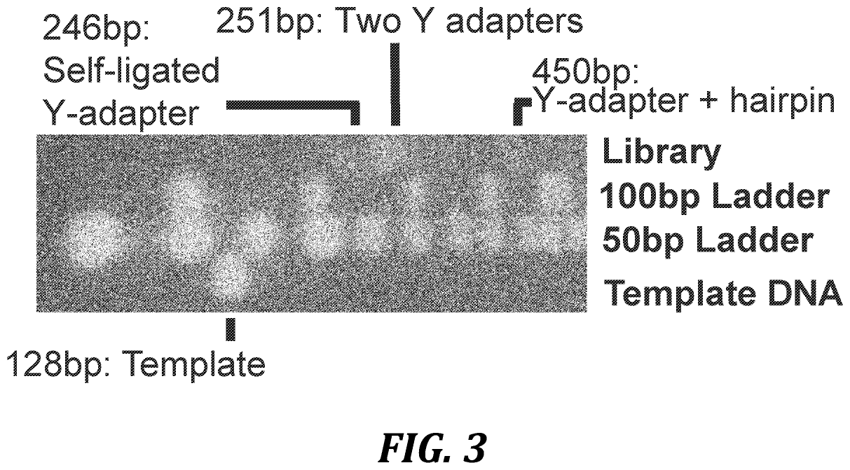

The most powerful form of UMID-mediated error correction is Duplex Sequencing (FIG. 1, Conventional Duplex), wherein each of the two strands in an individual DNA duplex are given a common label such that error-corrected consensus reads can be generated from each strand and subsequently compared to that of its mate to identify true mutations that are shared by both. Duplex Sequencing reduces error rates to ˜10⁻¹⁰ per base and can distinguish true coding mutations from DNA lesions (which are observed on only one strand). Duplex Sequencing is the most accurate form of sequencing developed to date.

The major drawback of all-label or UMID-mediated error correction methods, Duplex Sequencing included, is that an extensive number of sequence reads are required because each template molecule must be redundantly interrogated multiple times to enable consensus generation. Fragments interrogated only once (i.e., “singletons”) can account for over half of all reads generated but are not suitable for use in error correction.

Critically, experimental data indicate that each template must be independently sequenced an average of 6-10 times for optimal performance, meaning that the approach consumes an order of magnitude greater sequencing depth than for the same assay performed without error correction. This means that substantially fewer specimens or less of the genome can be interrogated per sequencing run, and that markedly higher costs per sample are required than by standard sequencing implementations. Various bioinformatic methods to “rescue” otherwise unusable singletons and library preparation modifications have been proposed, but only modestly improve efficiency of the process. Because these requirements exceed the scale and operating budget of most clinical laboratories, label-mediated error correction techniques have seen little uptake in routine clinical practice. Rare clinical groups have adopted error corrected sequencing in niche applications where the benefits of the approach outweigh the financial and practical drawbacks, but the vast potential of error correction to improve performance of existing clinical assays remains largely unfulfilled.

An ideal error correction technology would retain the favorable properties of Duplex Sequencing (ultrasensitivity, quantitative precision, and resolution of amplifiable DNA lesions), while addressing its deficiencies by also providing: (1) scalability—the ideal approach will interrogate large numbers of genomic targets (i.e., from a few genes to the entire exome or genome); (2) cost-effectiveness—the total cost from specimen to result, including sequencing costs, must be inexpensive enough for routine use; and (3) efficiency—the ideal approach requires a minimal number of sequencing reads for compatibility with low-to-mid throughput sequencing platforms available to most clinical laboratories.

### Linked Duplex Sequencing

As further described in the Example Section, a novel experimental framework, Linked Duplex Sequencing implements these properties. Linked Duplex Sequencing is a sequencing strategy wherein the complementary sense and antisense strands of a double-stranded nucleic acid (e.g., DNA) molecule are physically joined by a linker adapter. The resulting duplex provides a single molecule template for sequencing that includes both the sense strand and complementary antisense strand sequence. Having the resulting complementary sequences produced from a single template permits comparison of each sequence (i.e., self-correct their sequences) to resolve true, biological mutations from sequencing errors or other artifacts. The approach eliminates the need for redundant sequencing of template molecules and is compatible with extant short read sequencing platforms (e.g., Illumina) already in widespread clinical use.

In accordance with the foregoing, in one aspect the disclosure provides for a method to prepare an error-corrected nucleic acid library for sequencing, the method can comprise: providing a double stranded nucleic acid molecule, comprising a positive strand and a negative strand, wherein the positive strand and the negative strand are substantially complementary, and wherein the double stranded nucleic acid molecule has a first end and a second end; covalently attaching a first sequencing adapter to the positive strand at the first end of the double stranded nucleic acid molecule; covalently attaching a second sequencing adapter to the negative strand at the first end of the double stranded nucleic acid molecule; and covalently attaching a first end of a single hairpin adapter to the positive strand at the second end of the double stranded nucleic acid molecule and covalently attaching a second end of the single hairpin adapter to the negative strand at the second end of the double stranded nucleic acid molecule, wherein the single hairpin adapter covalently joins the positive strand and the negative strand into a single covalently linked duplex strand for self-correction of sequencing errors.

### Library Preparation

In some embodiments, the method discloses attaching an adapter, a unique molecule identifier (UMID), and an index sequence to each amplicon or product generated by the method described above.

As used herein, an “adapter” is a sequence that permits universal amplification. A key feature of the adapter is to enable the unique amplification of the amplicon or product only without the need to remove existing template nucleic acid or purify the amplicons or products. This feature enables an “add only” reaction with fewer steps and ease of automation. The adapter is attached to the 5′ and 3′ end of the amplicon or product. The adapter may be Y-shaped, U-shaped, hairpin-shaped, or a combination thereof. In a specific embodiment, the adapter is Y-shaped. In an exemplary embodiment, the adapter may be an Illumina adapter for Illumina sequencing.

As used herein, a “UMID” is composed of random nucleotides to generate a complexity of random components far greater than the number of unique amplicons or products to be sequenced. This ensures that having the same random component attached to multiple amplicons or products is an extremely statistically improbable event. This complexity can easily be expanded by increasing the length of the random regions in the UMID. In some embodiments, the UMID can be about 5 to about 100 nucleotides. In other embodiments, the UMID can be about 10 to about 25 nucleotides (e.g., about 15 to about 20 nucleotides). In still other embodiments, the UMID is about 16 to about 18 nucleotides. In still other embodiments, the UMID can be 10, 11, 12, 13, 14, 15, 16, 17, 18, 19, 20, 21, 22, 23, 24 or 25 or more nucleotides. The UMID can be attached to the 5′ or 3′ end of the amplicon or product. In still other embodiments, the UMID can be attached to the 5′ end of the amplicon or product. In still other embodiments, the UMID can be within the hairpin adapter.

In some embodiments, an index sequence can also be attached to each amplicon or product generated. The addition of an index sequence allows pooling of multiple samples into a single sequencing run. This greatly increases experimental scalability, while maintaining extremely low error rates and conserving read length. The index sequence can be about 5 to about 10 nucleotides. Accordingly, the index sequence can be 5, 6, 7, 8, 9 or 10 or more nucleotides. In an embodiment, the index sequence is about 6 nucleotides.

In some embodiments, an adapter, a UMID, and an index sequence can be attached to each amplicon or product. In some embodiments, a nucleotide sequence comprising an adapter and a UMID can be attached to the 5′ end of each amplicon or product and a nucleotide sequence comprising an adapter and an index sequence can be attached to the 3′ end. In other embodiments, a nucleotide sequence comprising an adapter and a UMID can be attached to the 3′ end of each amplicon or product and a nucleotide sequence comprising an adapter and an index sequence can be attached to the 5′ end. In still other embodiments, a nucleotide sequence comprising an adapter, a UMID, and an index sequence can be attached to the 5′ end and a nucleotide sequence comprising an adapter can be attached to the 3′ end. In still yet another embodiment, a nucleotide sequence comprising an adapter, a UMID, and an index sequence is attached to the 3′ end and a nucleotide sequence comprising an adapter is attached to the 5′ end. In still other embodiments, a nucleotide sequence comprising an adapter and an index sequence can be attached to the 5′ end and a nucleotide sequence comprising an adapter and an index sequence can be attached to the 3′ end.

The nucleotide sequence comprising an adapter, a UMID, and/or an index sequence can be attached to the amplicon or product via methods known in the art. In certain embodiments, the nucleotide sequence comprising an adapter, a UMID, and/or an index sequence is ligated to an amplicon or product via methods standard in the art

In still other embodiments, the amplicon or product can further comprise a hairpin adapter.

### Hairpin Adapter

The hairpin adapter is an adapter that is capable of linking the two strands of the double stranded molecule. The hairpin adapter can covalently link the two strands of the double stranded molecule. The hairpin adapter can be anything that is capable of linking the two strands of the double stranded molecule, wherein the linked strands are formed into a single covalently linked duplex strand for self-correction of sequencing errors. See e.g., FIG. 1. Suitable hairpin adapters include, but are not limited to a nucleic acid molecule, including but not limited to DNA and RNA. In some embodiments, the hair pin adapter can include modified DNA (such as abasic DNA), RNA, PNA, LNA or PEG. In still other embodiments, the hairpin adapter can include a polymeric linker, a chemical linker, a polynucleotide, or a polypeptide. As used herein, the term “hairpin adapter” and any grammatical variations refer to a duplex formed by a single-stranded nucleic acid that doubles back on itself to form a double stranded region maintained by base-pairing between complementary base sequences on the same strand. In other embodiments, the hairpin adapter can comprise a hairpin loop region formed by unpaired bases. In still other embodiments, the hairpin sequence is located in an opposite end of the double-stranded DNA molecules with respect to the location of the double-stranded DNA adapter in the double-stranded DNA molecules.

In some embodiments, the single hairpin adapter is a partially double stranded nucleic acid molecule that has a secondary structure comprising a double stranded stem domain and a loop domain. In some embodiments, the stem domain comprises each end of the hairpin adapter to covalently attach to the positive end and the negative end of the second end of the double stranded nucleic acid molecule. In still other embodiments, the hairpin adapter can comprise a double stranded stem domain and a loop domain.

In still other embodiments, a hairpin adapter can include two complementary nucleic acid segments separated by a stretch of non-complementary nucleotides. In some embodiments, the structure of the hair pin adapter can include a double-stranded stem formed by the complementary segments and a single-stranded loop. In some embodiments, the stem can be blunt ended. In other embodiments, the stem can include a 5′ single-stranded overhang. In still other embodiments, the stem can include a 3′ single-stranded overhang. It will be evident that where the hairpin adapter is to ligate to a blunt end of the fragment (e.g., a product fragment produced by digestion with a restriction endonuclease that leaves blunt ends, or a product produced by digestion with a restriction endonuclease that leaves a single-stranded overhang followed by polishing with a polymerase to fill in a 5′ overhang or remove a 3′ overhang), the hairpin adapter is preferably blunt ended. In other embodiments, where the hairpin adapter is to ligate to a fragment having an overhang, the hairpin adapter preferably has a complementary overhang, e.g., a single-stranded overhang that is complementary to a single-stranded overhang on the product fragment.

Suitable hairpin adapters are readily designed and synthesized using conventional nucleic acid synthesis techniques. In some embodiments, the hairpin adapter(s) can be present during the restriction digestion or can be added subsequently to the reaction mixture. In other embodiments, the hairpin adapter(s) are typically provided in excess, e.g., to speed the reaction and to discourage re-ligation between the product fragment and the loop regions removed from it by the restriction enzyme(s). In still other embodiments, the hairpin adapter can be linked to the double stranded nucleic acid molecule by any suitable means known in the art. In other embodiments, the hairpin adapter can be synthesized separately and chemically attached or enzymatically ligated to the double stranded nucleic acid.

In some embodiments, the hairpin adapter can be covalently linked at or near the positive strand and/or the negative strand of the second end of the double stranded nucleic acid molecule. In other embodiments, the hairpin adapter can be covalently linked within 10 nucleotides of the end of the positive strand and/or the negative strand of the second end of the double stranded nucleic acid molecule.

In some embodiments, wherein the single hairpin adapter comprises a number (N) of nucleotides. In some embodiments, N is an integer selected from 6 to 300. In some embodiments, the hairpin adapter can comprise at least 6 nucleotides. In some embodiments, the hairpin adapter can comprise at least 10 nucleotides. In some embodiments, the hairpin adapter can comprise at least 20 nucleotides. In some embodiments, the hairpin adapter can comprise at least 30 nucleotides. In some embodiments, the hairpin adapter can comprise at least 40 nucleotides. In some embodiments, the hairpin adapter can comprise at least 50 nucleotides. In some embodiments, the hairpin adapter can comprise at least 60 nucleotides. In some embodiments, the hairpin adapter can comprise at least 70 nucleotides. In some embodiments, the hairpin adapter can comprise at least 80 nucleotides. In some embodiments, the hairpin adapter can comprise at least 90 nucleotides. In some embodiments, the hairpin adapter can comprise at least 100 nucleotides. In some embodiments, the hairpin adapter can comprise at least 125 nucleotides. In some embodiments, the hairpin adapter can comprise at least 150 nucleotides. In some embodiments, the hairpin adapter can comprise at least 175 nucleotides. In some embodiments, the hairpin adapter can comprise at least 200 nucleotides. In some embodiments, the hairpin adapter can comprise at least 225 nucleotides. In some embodiments, the hairpin adapter can comprise at least 250 nucleotides. In some embodiments, the hairpin adapter can comprise at least 275 nucleotides. In some embodiments, the hairpin adapter can comprise at least 300 nucleotides.

In some embodiments, each nucleotide comprising the hairpin adapter can be selected independently. In some embodiments, the nucleotide sequence can be completely random, wherein each sequence position may be any nucleotide, (i.e., each position can be an adenine (A), cytosine (C), guanine (G), thymine (T), or uracil (U)) or any other natural or non-natural DNA or RNA nucleotide or nucleotide-like substance or analog with base-pairing properties (e.g., xanthosine, inosine, hypoxanthine, xanthine, 7-methylguanine, 7-methylguanosine, 5,6-dihydrouracil, 5-methylcytosine, dihydrouridine, isocytosine, isoguanine, deoxynucleosides, nucleosides, peptide nucleic acids, locked nucleic acids, glycol nucleic acids and threose nucleic acids). In other embodiments, the nucleotide sequence can be semi-random, wherein a known sequence of N length is combined with a random sequence of N length to make the full-length hairpin adapter. In still other embodiments, the nucleotide sequence can be non-random, wherein the full-length hairpin adapter comprises a known sequence.

In some embodiments, the hairpin adapter can comprise a unique molecule identifier (UMID) sequence. In still other embodiments, the loop domain of the hairpin adapter can comprise a UMID sequence. In some embodiments, the loop domain of the hairpin adapter can comprise a secondary index sequence adjacent to the UMID sequence.

### Sample

In some embodiments, the method described above can be employed to analyze genomic DNA from virtually any organism, including, but not limited to, plants, animals (e.g., reptiles, mammals, insects, worms, fish, etc.), tissue samples, bacteria, fungi (e.g., yeast), phage, viruses, cadaveric tissue, archaeological/ancient samples, etc. In some embodiments, the genomic DNA used in the method can be derived from a mammal. In some embodiments, the mammal is a human. In other embodiments, the sample can contain genomic DNA from a mammalian cell, such as, a human, mouse, rat, or monkey cell. In other embodiments, the sample can be made from cultured cells or cells of a clinical sample, e.g., a tissue biopsy, scrape or lavage or cells of a forensic sample (i.e., cells of a sample collected at a crime scene). In still other embodiments, the nucleic acid sample can be obtained from a biological sample such as cells, tissues, bodily fluids, and stool. In some embodiments, the bodily fluids of interest include but are not limited to, blood, serum, plasma, saliva, mucous, phlegm, cerebral spinal fluid, pleural fluid, tears, lacteal duct fluid, lymph, sputum, synovial fluid, urine, amniotic fluid, and semen. In some embodiments, a sample can be obtained from a subject, e.g., a human. In some embodiments, the sample comprises fragments of human genomic DNA. In some embodiments, the sample can be obtained from a cancer patient. In some embodiments, the sample can be made by extracting fragmented DNA from a patient sample, e.g., a formalin-fixed paraffin embedded tissue sample. In some embodiments, the patient sample can be a sample of cell-free “circulating” DNA from a bodily fluid, e.g., peripheral blood e.g., from the blood of a patient or of a pregnant female. The DNA fragments used in the initial step of the method should be non-amplified DNA that has not been denatured beforehand.

In still other embodiments, the DNA in the initial sample can be made by extracting genomic DNA from a biological sample, and then fragmenting it. In some embodiments, the fragmenting can be done mechanically (e.g., by sonication, nebulization, or shearing, etc.) or using a double stranded DNA “dsDNA” fragmentase enzyme (New England Biolabs, Ipswich Mass.). In some of these methods (e.g., the mechanical and fragmentase methods), after the DNA is fragmented, the ends can be polished and A-tailed prior to ligation to one or more adapters. Alternatively, the ends can be polished and ligated to adapters in a blunt-end ligation reaction. In still other embodiments, double stranded nucleic acid molecules can be produced by transposon mediated fragmentation. In other embodiments, the DNA in the initial sample can already be fragmented (e.g., as is the case for formalin-fixed paraffin-embedded tissue (FPET) samples and circulating cell-free DNA (cfDNA), e.g., ctDNA). The fragments in the initial sample can have a median size that is below 1 kb (e.g., in the range of 50 bp to 500 bp, or 80 bp to 400 bp), although fragments having a median size outside of this range can be used.

In some embodiments, the amount of DNA in a sample can be limiting. For example, the initial sample of fragmented DNA can contain less than 200 ng of fragmented human DNA, (e.g., 1 pg to 20 pg, 10 pg to 200 ng, 100 μg to 200 ng, 1 ng to 200 ng or 5 ng to 50 ng), or less than 10,000 (e.g., less than 5,000, less than 1,000, less than 500, less than 100, less than 10 or less than 1) haploid genome equivalents, depending on the genome.

In some embodiments, sample identifiers (i.e., a sequence that identifies the sample to which the sequence is added, which can identify the patient, or a tissue, etc.) can be added to the polynucleotides prior to sequencing, so that multiple (e.g., at least 2, at least 4, at least 8, at least 16, at least 48, at least 96 or more) samples can be multiplexed. In these embodiments, the sample identifier ligated can be to the initial polynucleotides as part of the asymmetric adapter, or the sample identifier can be ligated to the polynucleotides in the sub-samples, before or after amplification of those polynucleotides. Alternatively, the tag can be added by primer extension, i.e., using a primer that has a 3′ end that hybridizes to an adapter sequence, and a 5′ tail that contains the sample identifier.

In some embodiments, the double stranded nucleic acid molecule can be generated by shearing a larger double stranded nucleic acid molecule. In other embodiments, the double stranded nucleic acid molecule can be generated by enzymatically fragmenting a larger double stranded nucleic acid molecule. In some embodiments, the double stranded nucleic acid molecule has an overhang end. In other embodiments, the double stranded nucleic acid molecule has a blunt end. In still other embodiments, the double stranded nucleic acid molecule is generated by transposon mediated fragmentation.

In some embodiments, the method comprises adding one or more adenine residues at a 3′ end of the positive strand and/or adding one or more adenine residues at a 3′ end of the negative strand.

### Sequencing

In some embodiments, the sequencing step can be done using any convenient next generation sequencing method and can result in at least 10,000, at least 50,000, at least 100,000, at least 500,000, at least 1M at least 10M at least 100M or at least 1B sequence reads. In some cases, the reads are paired-end reads. In some embodiments, the sequencing can be done using an Illumina platform. However, the sequencing and related methods can be adapted to other sequencing platforms that use long single reads or shorter paired-end reads as well-known to one of ordinary skill in the art.

In some embodiments, the primers used for amplification can be compatible with use in any next generation sequencing platform in which primer extension is used, e.g., Illumina's reversible terminator method, Roche's pyrosequencing method (454), Life Technologies sequencing by ligation (the SOLID platform), Life Technologies' Ion Torrent platform or Pacific Biosciences' fluorescent base-cleavage method. Examples of such methods are described in the following references: Margulies et al., (Nature 2005 437: 376-80): Ronaghi et al., (Analytical Biochemistry 1996 242: 84-9): Shendure (Science 2005 309: 1728); Imelfort et al., (Brief Bioinform. 2009 10:609-18); Fox et al., (Methods Mol Biol. 2009: 553:79-108): Appleby et al., (Methods Mol Biol. 2009; 513:19-39) English (PLOS One. 2012 7: e47768) and Morozova (Genomics. 2008 92:255-64), which are incorporated by reference for the general descriptions of the methods and the particular steps of the methods, including all starting products, reagents, and final products for each of the steps. In still other embodiments, the sequencing can be done by paired-end sequencing, although single read sequencing can be done in some cases.

In still other embodiments, the method comprises sequencing at least one covalently linked duplex strand amplicons to produce at least one sequence read comprising a first subsequence corresponding to at least a portion of the positive strand of the double stranded nucleic acid molecule and a second subsequence corresponding to at least a portion of the negative strand of the double stranded nucleic acid molecule. In some embodiments, only the first subsequence and/or the second subsequence with a unique UMID sequence is analyzed. In still other embodiments, the analysis comprises comparing the sequence of the first subsequence to the sequence of the second subsequence and a variation observed in both the first subsequence and the second subsequence is a genetic variation. In some embodiments, there can be 1 variation observed in both the first subsequence and the second subsequence. In other embodiments, there could be 2 or more variations observed in both the first subsequence and the second subsequence. In some embodiments, there could be 3 or more variations observed in both the first subsequence and the second subsequence. In some embodiments, there could be 4 or more variations observed in both the first subsequence and the second subsequence. In some embodiments, there could be 5 or more variations observed in both the first subsequence and the second subsequence. In some embodiments, there could be 6 or more variations observed in both the first subsequence and the second subsequence. In some embodiments, there could be 7 or more variations observed in both the first subsequence and the second subsequence. In some embodiments, there could be 8 or more variations observed in both the first subsequence and the second subsequence. In some embodiments, there could be 9 or more variations observed in both the first subsequence and the second subsequence. In some embodiments, there could be 10 or more variations observed in both the first subsequence and the second subsequence.

In still other embodiments, the analysis comprises comparing the sequence of the first subsequence to the sequence of the second subsequence and a variation mismatch between the first subsequence and the second subsequence is a sequencing error. In some embodiments, there can be 1 variation mismatch between the first subsequence and the second subsequence. In some embodiments, there can be 2 or more variation mismatches between the first subsequence and the second subsequence. In some embodiments, there can be 3 or more variation mismatches between the first subsequence and the second subsequence. In some embodiments, there can be 4 or more variation mismatches between the first subsequence and the second subsequence. In some embodiments, there can be 5 or more variation mismatches between the first subsequence and the second subsequence. In some embodiments, there can be 6 or more variation mismatches between the first subsequence and the second subsequence. In some embodiments, there can be 7 or more variation mismatches between the first subsequence and the second subsequence. In some embodiments, there can be 8 or more variation mismatches between the first subsequence and the second subsequence. In some embodiments, there can be 9 or more variation mismatches between the first subsequence and the second subsequence. In some embodiments, there can be 10 or more variation mismatches between the first subsequence and the second subsequence.

In some embodiments, the double stranded nucleic acid molecule is a double stranded DNA molecule. In other embodiments, the method comprises amplifying the single covalently linked duplex strand to produce a plurality of covalently linked duplex strand amplicons. In still other embodiments, the method further comprises preparing a plurality of double stranded nucleic acid molecules for sequencing, by performing the method described above a plurality of times for different double stranded nucleic acid molecules using a plurality hairpin adapters comprising different UMID sequences.

In another aspect, the disclosure provides for a linked duplex nucleic acid molecule produced by the method described above.

In another aspect, the disclosure provides for method for detecting one or more genetic variants in a biological sample, the method comprising: generating a sequencing library by performing the method as described above, wherein the sequencing library comprises a plurality of covalently linked duplex strands each comprising a unique UMID sequence; amplifying at least a portion of the covalently linked duplex strands to produce an amplified sequencing library comprising a plurality of copies of the covalently linked duplex strands; sequencing at least a portion of the covalently linked duplex strands to obtain at least one sequence read comprising a first subsequence corresponding to at least a portion of the positive strand of the double stranded nucleic acid molecule and a second subsequence corresponding to at least a portion of the negative strand of the double stranded nucleic acid molecule; and detecting a presence or absence of one or more genetic variants in the biological sample, by comparing the sequence of the first subsequence to the sequence of the second subsequence, wherein one or more variants observed in both subsequences are genetic variants as described above.

In some embodiments, a mismatch of one or more variants between the first subsequence and the second subsequence is a sequencing error as described above. In still other embodiments, the method further comprises producing the double stranded nucleic acid molecule by transposon mediated fragmentation.

In another aspect, the disclosure provides for a kit comprising: a first sequencing adapter, a second sequencing adapter, a single hairpin adapter, one or more primers that hybridize to sequences in the first sequencing adapter and/or second sequencing adapter, or a complement thereof, and free nucleotides (dNTPs), a DNA polymerase, a ligase, and written indicia instructing the performance of the method described above.

In another aspect, the disclosure provides for a kit comprising: a first sequencing adapter, a second sequencing adapter, a single hairpin adapter, a transposome, one or more primers that hybridize to a transposon sequence, a DNA polymerase, a ligase, and written indicia instructing the performance of the method described above.

In some embodiments, the various components of the kit can be present in separate containers or certain compatible components may be pre-combined into a single container, as desired. In other embodiments, the written indicia (i.e., instructions) for practicing the subject methods are generally recorded on a suitable recording medium. For example, the instructions may be printed on a substrate, such as paper or plastic, etc. In other embodiments, the instructions may be present in the kits as a package insert, in the labeling of the container of the kit or components thereof (i.e., associated with the packaging or subpackaging) etc. In still other embodiments, the instructions are present as an electronic storage data file present on a suitable computer readable storage medium, e.g., CD-ROM, diskette, etc. In vet other embodiments, the actual instructions are not present in the kit, but means for obtaining the instructions from a remote source, e.g., via the internet, are provided. An example of this embodiment is a kit that includes a web address where the instructions can be viewed and/or from which the instructions can be downloaded. As with the instructions, this means for obtaining the instructions is recorded on a suitable substrate.

In some embodiments, the kit comprises a single hairpin adapter is a partially double stranded nucleic acid molecule that has a secondary structure comprising a double stranded stem domain and a loop domain, wherein the stem domain comprises each end of the hairpin adapter to covalently attach to the positive end and the negative end of the second end of the double stranded nucleic acid molecule. In still other embodiments, the kit comprises a single hairpin adapter comprises a number (N) of nucleotides, wherein each nucleotide is selected independently, and wherein N is an integer selected from 6 to 100. In still other embodiments, the kit comprises a loop domain comprises a unique molecule identifier (UMID) sequence. In still other embodiments, the kit comprises a loop domain comprises a secondary index sequence adjacent to the UMID sequence.

### Additional Definitions

Unless Specifically Defined Herein, all Terms Used Herein have the Same Meaning as they would to one skilled in the art of the present disclosure. Practitioners are particularly directed to Ausubel, F. M., et al. (eds.), Current Protocols in Molecular Biology, John Wiley & Sons, New York (2010), Coligan, J. E., et al. (eds.), Current Protocols in Immunology, John Wiley & Sons, New York (2010), Mirzaei, H. and Carrasco, M. (eds.), Modern Proteomics-Sample Preparation, Analysis and Practical Applications in Advances in Experimental Medicine and Biology, Springer International Publishing, 2016, and Comai, L, et al., (eds.), Proteomic; Methods and Protocols in Methods in Molecular Biology, Springer International Publishing, 2017, for definitions and terms of art.

The use of the term “or” in the claims is used to mean “and/or” unless explicitly indicated to refer to alternatives only or the alternatives are mutually exclusive, although the disclosure supports a definition that refers to only alternatives and “and/or.”

The words “a” and “an,” when used in conjunction with the word “comprising” in the claims or specification, denotes one or more, unless specifically noted.

Unless the context clearly requires otherwise, throughout the description and the claims, the words “comprise,” “comprising,” and the like, are to be construed in an inclusive sense as opposed to an exclusive or exhaustive sense, which is to indicate, in the sense of “including, but not limited to.” Words using the singular or plural number also include the plural and singular number, respectively. The word “about” indicates a number within range of minor variation above or below the stated reference number. For example, “about” can refer to a number within a range of 10%, 9%, 8%, 7%, 6%, 5%, 4%, 3%, 2%, or 1% above or below the indicated reference number.

As used herein, the term “nucleic acid” refers to a polymer of nucleotide monomer units or “residues”. The nucleotide monomer subunits, or residues, of the nucleic acids each contain a nitrogenous base (i.e., nucleobase) a five-carbon sugar, and a phosphate group. The identity of each residue is typically indicated herein with reference to the identity of the nucleobase (or nitrogenous base) structure of each residue. Canonical nucleobases include adenine (A), guanine (G), thymine (T), uracil (U) (in RNA instead of thymine (T) residues) and cytosine (C).

The five-carbon sugar to which the nucleobases are attached can vary depending on the type of nucleic acid. For example, the sugar is deoxyribose in DNA and is ribose in RNA. In some instances herein, the nucleic acid residues can also be referred with respect to the nucleoside structure, such as adenosine, guanosine, 5-methyluridine, uridine, and cytidine. Moreover, alternative nomenclature for the nucleoside also includes indicating a “ribo” or deoxyribo” prefix before the nucleobase to infer the type of five-carbon sugar. For example, “ribocytosine” as occasionally used herein is equivalent to a cytidine residue because it indicates the presence of a ribose sugar in the RNA molecule at that residue. The nucleic acid polymer can be or comprise a deoxyribonucleotide (DNA) polymer, a ribonucleotide (RNA) polymer, including mRNA. The nucleic acids can also be or comprise a PNA polymer, or a combination of any of the polymer types described herein (e.g., contain residues with different sugars).

The term “sample” as used herein relates to a material or mixture of materials, typically containing one or more analytes of interest. In one embodiment, the term as used in its broadest sense, refers to any plant, animal, microbial or viral material containing genomic DNA, such as, for example, tissue or fluid isolated from an individual (including without limitation plasma, serum, cerebrospinal fluid, lymph, tears, saliva, and tissue sections) or from in vitro cell culture constituents, as well as samples from the environment.

The term “nucleic acid sample,” as used herein, denotes a sample containing nucleic acids. Nucleic acid samples used herein can be complex in that they contain multiple different molecules that contain sequences. Genomic DNA samples from a mammal (e.g., mouse or human) are types of complex samples. Complex samples can have more than about 10⁴, 10⁵, 10⁶ or 10⁷, 10⁸, 10⁹ or 10¹⁰ different nucleic acid molecules. A DNA target can originate from any source such as genomic DNA, or an artificial DNA construct. Any sample containing nucleic acid, e.g., genomic DNA from tissue culture cells or a sample of tissue, can be employed herein.

The term “nucleic acid” and “polynucleotide” are used interchangeably herein to describe a polymer of any length, e.g., greater than about 2 bases, greater than about 10 bases, greater than about 100 bases, greater than about 500 bases, greater than 1000 bases, greater than 10,000 bases, greater than 100,000 bases, greater than about 1,000,000, up to about 10¹⁰ or more bases composed of nucleotides, e.g., deoxyribonucleotides or ribonucleotides, and can be produced enzymatically or synthetically which can hybridize with naturally occurring nucleic acids in a sequence specific manner analogous to that of two naturally occurring nucleic acids, e.g., can participate in Watson-Crick base pairing interactions. Naturally-occurring nucleotides include guanine, cytosine, adenine, thymine, uracil (G, C, A, T and U respectively). DNA and RNA have a deoxyribose and ribose sugar backbone, respectively, whereas PNA's backbone is composed of repeating N-(2-aminoethyl)-glycine units linked by peptide bonds.

“Primer” means an oligonucleotide, either natural or synthetic, that is capable, upon forming a duplex with a polynucleotide template, of acting as a point of initiation of nucleic acid synthesis and being extended from its 3′ end along the template so that an extended duplex is formed. The sequence of nucleotides added during the extension process is determined by the sequence of the template polynucleotide. Usually, primers are extended by a DNA polymerase. Primers are generally of a length compatible with their use in synthesis of primer extension products and are usually in the range of between 8 to 100 nucleotides in length, such as 10 to 75, 15 to 60, 15 to 40, 18 to 30, 20 to 40, 21 to 50, 22 to 45, 25 to 40, and so on. Typical primers can be in the range of between 10-50 nucleotides long, such as 15-45, 18-40, 20-30, 21-25 and so on, and any length between the stated ranges. In some embodiments, the primers are usually not more than about 10, 12, 15, 20, 21, 22, 23, 24, 25, 26, 27, 28, 29, 30, 35, 40, 45, 50, 55, 60, 65, or 70 nucleotides in length. In some embodiments a primer can be activated prior to primer extension. For example, some primers have a 3′ block and internal RNA base. The RNA base can be removed by RNaseH or another treatment, thereby producing a 3′ hydroxyl group which can be extended. Other methods for activating primers exist.

Primers are usually single-stranded for maximum efficiency in amplification but can alternatively be double-stranded or partially double-stranded. If double-stranded, the primer is usually first treated to separate its strands before being used to prepare extension products. This denaturation step is typically effected by heat, but can alternatively be carried out using alkali, followed by neutralization.

Thus, a “primer” is complementary to a template, and complexes by hydrogen bonding or hybridization with the template to give a primer/template complex for initiation of synthesis by a polymerase, which is extended by the addition of covalently bonded bases linked at its 3′ end complementary to the template in the process of DNA synthesis.

The term “hybridization” or “hybridizes” refers to a process in which a region of nucleic acid strand anneals to and forms a stable duplex, either a homoduplex or a heteroduplex, under normal hybridization conditions with a second complementary nucleic acid strand and does not form a stable duplex with unrelated nucleic acid molecules under the same normal hybridization conditions. The formation of a duplex is accomplished by annealing two complementary nucleic acid strand region in a hybridization reaction. The hybridization reaction can be made to be highly specific by adjustment of the hybridization conditions (often referred to as hybridization stringency) under which the hybridization reaction takes place, such that two nucleic acid strands will not form a stable duplex, e.g., a duplex that retains a region of double-strandedness under normal stringency conditions, unless the two nucleic acid strands contain a certain number of nucleotides in specific sequences which are substantially or completely complementary. “Normal hybridization or normal stringency conditions” are readily determined for any given hybridization reaction. See, e.g., Ausubel et al., Current Protocols in Molecular Biology, John Wiley & Sons, Inc., New York, or Sambrook et al., Molecular Cloning: A Laboratory Manual, Cold Spring Harbor Laboratory Press. As used herein, the term “hybridizing” or “hybridization” refers to any process by which a strand of nucleic acid binds with a complementary strand through base pairing.

The term “amplifying” as used herein refers to the process of synthesizing nucleic acid molecules that are complementary to one or both strands of a template nucleic acid. Amplifying a nucleic acid molecule can include denaturing the template nucleic acid, annealing primers to the template nucleic acid at a temperature that is below the melting temperatures of the primers, and enzymatically elongating from the primers to generate an amplification product. The denaturing, annealing and elongating steps each can be performed one or more times. In certain cases, the denaturing, annealing, and elongating steps are performed multiple times such that the amount of amplification product is increasing, often times exponentially, although exponential amplification is not required by the present methods. Amplification typically requires the presence of deoxyribonucleoside triphosphates, a DNA polymerase enzyme, and an appropriate buffer and/or co-factors for optimal activity of the polymerase enzyme. The term “amplification product” refers to the nucleic acids, which are produced from the amplifying process as defined herein.

The terms “determining,” “measuring,” “evaluating,” “assessing,” “assaying,” and “analyzing” are used interchangeably herein to refer to any form of measurement and include determining if an element is present or not. These terms include both quantitative and/or qualitative determinations. Assessing can be relative or absolute. “Assessing the presence of” includes determining the amount of something present, as well as determining whether it is present or absent.

The term “ligating,” as used herein, refers to the enzymatically catalyzed joining of the terminal nucleotide at the 5′ end of a first DNA molecule to the terminal nucleotide at the 3′ end of a second DNA molecule.

The term “strand” as used herein refers to a nucleic acid made up of nucleotides covalently linked together by covalent bonds, e.g., phosphodiester bonds. In a cell, DNA usually exists in a double-stranded form, and as such, has two complementary strands of nucleic acid referred to herein as the “Watson” (or “TOP”) and “Crick” (or “BOT”) strands. In certain cases, complementary strands of a chromosomal region can be referred to as “plus” and “minus” strands, the “first” and “second” strands, the “coding” and “noncoding” strands, the “top” and “top” strands, “positive” and “negative” strands, or the “sense” and “antisense” strands. The assignment of a strand as being a Watson (or “TOP”) or Crick (or BOT) strand is arbitrary and does not imply any particular orientation, function, or structure.

The term “extending”, as used herein, refers to the extension of a primer by the addition of nucleotides using a polymerase. If a primer that is annealed to a nucleic acid is extended, the nucleic acid acts as a template for extension reaction.

The term “sequencing,” as used herein, refers to a method by which the identity of at least 10 consecutive nucleotides (e.g., the identity of at least 20, at least 50, at least 100 or at least 200 or more consecutive nucleotides) of a polynucleotide is obtained.

The terms “next-generation sequencing” or “high-throughput sequencing”, as used herein, refer to the so-called parallelized sequencing-by-synthesis or sequencing-by-ligation platforms currently employed by Illumina, Life Technologies, and Roche, etc. Next-generation sequencing methods can also include nanopore sequencing methods such as that commercialized by Oxford Nanopore Technologies, electronic-detection based methods such as Ion Torrent technology commercialized by Life Technologies, or single-molecule fluorescence-based methods such as that commercialized by Pacific Biosciences.

The terms “sample identifier sequence” or “sample index” refer to a type of barcode that can be appended to a target polynucleotide, where the sequence identifies the source of the target polynucleotide (i.e., the sample from which sample the target polynucleotide is derived). In use, each sample is tagged with a different sample identifier sequence (e.g., one sequence is appended to each sample, where the different samples are appended to different sequences), and the tagged samples are pooled. After the pooled sample is sequenced, the sample identifier sequence can be used to identify the source of the sequences.

The term “sequencing adapter” refers to a nucleic acid molecule that can be joined to at least one strand of a double-stranded DNA molecules for use in priming PCR or sequencing. The sequencing adapter molecule can be at least partially double-stranded and the sequencing adapter can be 20 to 150 bases in length, e.g., 40 to 120 bases, although adapters with base lengths outside of this range are possible. The sequencing adapters typically include (from 5′ to 3′) a first region, e.g., of about 10-15, e.g., 12, nucleotides; a second region, e.g., of about 20-60, e.g., 40, nucleotides that forms at least one (and preferably only one) hairpin loops and includes a sequence suitable for use in PCR priming and/or sequencing, e.g., next generation sequencing (NGS), flanked by at least one (and preferably only one) uracil; and a third region, e.g., of about 10-15, e.g., 13, nucleotides that is complementary to the first region. The lengths of the first, second and third regions can vary depending on the NGS method selected, as they are dependent on the sequences that are necessary for priming for use with the selected NGS platform. In some embodiments, commercially available adapters that are variations of standard adapters (e.g., from Illumina or NEB) can be used.

The term “amplification error” refers to a mis-incorporated base, or a deletion/insertion caused by polymerase stutter. Stutter usually occurs in repeat sequences, e.g., short tandem repeats (STRs) or microsatellite repeats and is presumed to be due to miscopying or slippage by the polymerase.

The term “duplex sequencing” refers to a method in which sequences for both strands of a double-stranded molecule of genomic DNA are obtained. In duplex sequencing, the sequences derived from the top strand of double-stranded molecule of genomic DNA are distinguishable from sequences derived from the bottom strand of that molecule in such a way that the sequences for the top and bottom strands from the same double-stranded molecule of genomic DNA can be compared. As used herein, a “subsequence” (i.e., subsequence of a particular sequence) is a sequence that can be derived from the parent sequence.

As used herein, “genetic variation” refers to a variation that occurs due to a conversion or change in genetic composition. The genetic variation may be an allele, a Single Nucleotide Polymorphism (SNP), a mutation, or combinations thereof. An allele is an alternative form of a gene which expresses a different phenotype while occupying the same locus of a given chromosome. An allele also refers to a gene which has a different nucleotide sequence while occupying the same locus in a homologous chromosome. A mutation may include a point mutation, a transition mutation, a transversion mutation, a missense mutation, a nonsense mutation, a duplication, a deletion, an insertion, a translocation, an inversion, or combinations thereof. SNP refers to a variation in one or a few nucleotides of a genomic sequence reflecting variations among individuals. A “variation” can include a genetic variation as described above (i.e., true biological variation). Additionally, a variation can also refer to a mismatch due to a sequences error and for this reason is not considered a true biological variation.

Disclosed are materials, compositions, and components that can be used for, can be used in conjunction with, can be used in preparation for, or are products of the disclosed methods and compositions. It is understood that, when combinations, subsets, interactions, groups, etc., of these materials are disclosed, each of various individual and collective combinations is specifically contemplated, even though specific reference to each and every single combination and permutation of these compounds cannot be explicitly disclosed. This concept applies to all aspects of this disclosure including, but not limited to, steps in the described methods. Thus, specific elements of any foregoing embodiments can be combined or substituted for elements in other embodiments. For example, if there are a variety of additional steps that can be performed, it is understood that each of these additional steps can be performed with any specific method steps or combination of method steps of the disclosed methods, and that each such combination or subset of combinations is specifically contemplated and should be considered disclosed. Additionally, it is understood that the embodiments described herein can be implemented using any suitable material such as those described elsewhere herein or as known in the art.

Publications cited herein and the subject matter for which they are cited are hereby specifically incorporated by reference in their entireties.

### Examples

The following examples are provided for the purpose of illustrating, not limiting, the disclosure.

This Example describes the linked duplex sequencing strategy (FIG. 1: right panel), wherein two strands of DNA are covalently joined from an initial template fragment into a single, covalently linked molecule, so that error correction of the duplex can be performed comparing the two linked strands.

### Introduction

Briefly, in one embodiment, sheared and A-tailed DNA is ligated to a standard Illumina Y-adapter (bearing i5 and i7 sequencing adapters) and a partially double-stranded “hairpin” adapter integrating a 12 bp unique molecular identifier (UMID). PCR converts the ligated product to a fully double stranded molecule linking the two strands of the original template. Paired end Illumina sequencing is performed, with each read interrogating one of the two original strands. The standard i7 index read is repurposed to interrogate the sequence of the UMID. Every read with a unique UMID is carried forward for analysis; redundant sequencing of molecules bearing the same UMID is not necessary. Any reads carrying the same UMID and having the same end-mapping position in the genome are deduplicated to avoid representational bias. The sequences of the two strands from each template molecule are compared, and only variation observed in both are accepted as true sequences (e.g., mutations).

The approach supports sample multiplexing by inclusion of a standard, sample-specific index sequence with the i5 adapter. Optionally, a secondary index adjacent to the UMID can be included, if dual-indexing is required. Although the inventor's pilot studies are intended for Illumina chemistries, the approach could be generalized to other sequencers that use long single reads or shorter paired-end reads.

### Estimation of Error

Because the disclosed strategy requires agreement of both DNA strands to verify potential mutations, the only false positives will result from paired errors that occur on one strand and the complementary position and sequence of the other strand. Spontaneous base substitution errors meeting this condition can therefore be estimated as (per base error rate)²/3. Adding the measured error rates of Illumina sequencing (0.1%-0.5% per base) with that of the high-fidelity polymerase used for library construction (5×10⁻⁷ per base), the cumulative error rate of the disclosed strategy is thereby approximated at 8.3×10⁻⁶ to 3.3×10⁻⁷ per base, or ˜1 in 120,000 to ˜1 in 3,000,000. This error rate is higher than what is achievable by conventional duplex sequencing (˜1 in 10⁹ errors), but it is (1)>3 orders of magnitude better than standard sequencing protocols, (2)>1 order of magnitude better than single-stranded UMID methods, and (3)>2 orders of magnitude more sensitive than is thought to be meaningful for ultrasensitive clinical applications like minimal residual detection.

Estimation of Sequencing requirements

The performance of the disclosed approach must also be considered with respect to its demands for sequencing power. The reads necessary for conventional duplex sequencing to sequence a target of a specified size at a predetermined depth include: (1) the length of the target being interrogated [T], (2) the depth required per base [D], (3) effective sequence read length into genomic DNA after reading through UMIs [R] (assuming 150 bp paired end reads, this equals 266 bp), and (4) how many copies of each individual template must be sequenced to achieve error correction [C] (6 is recommended). Read requirements to achieve 95% likelihood of obtaining sufficient copies of a molecule to achieve error reduction can be approximated by sampling with replacement, yielding the formula:

$$C ( \frac{log ( 1 - 0.95 )}{log ( 1 - \frac{1 / D \times T \times 2}{R}} ) .$$

This formula projects fewer reads than needed based on empiric observations, so should be considered conservative.

In contrast, redundant sequencing is not required for Linked Duplex, so read requirements are dictated only by (1) the length of the target being interrogated [T], (2) the depth required per base [D], and (3) sequence read length [R] (assumes 150 bp reads overlapping fully), yielding the formula:

$$\frac{D \times T}{R} .$$

Comparing these two projections for sequencing panels of various sizes (FIG. 2) shows that Linked Duplex sequencing requires <3% of the reads needed for conventional duplex sequencing, and only twice the number of reads needed for non-error corrected sequencing at equivalent depth. For context, this efficiency would enable users to sequence up to ten 1 Mb gene oncology panels to a depth of 6,000× on a single Nextseq500 run.

Proof of principle library construction and sequencing: To investigate the technical feasibility of the disclosed approach, sequencing libraries from a PCR amplicon of appropriate size (123 bp) were produced. The amplicon was ligated with a mixture of Y-adapter and custom hairpin adapter (71 bp), followed by PCR amplification. This resulted primarily in a 251 bp product (consistent with the amplicon receiving 2 Y-adapters) and products at 450 bp, the expected size from the fully converted template (FIG. 1, step c; FIG. 3). Size selection and paired-end sequencing of these products was successfully performed, both (a) confirming the identity of the molecule and (b) showing that the products is compatible with Illumina sequencing despite their strong secondary structure.

While illustrative embodiments have been illustrated and described, it will be appreciated that various changes can be made therein without departing from the spirit and scope of the invention.

1. A method to prepare an error-corrected nucleic acid library for sequencing, the method comprising:
providing a double stranded nucleic acid molecule, comprising a positive strand and a negative strand, wherein the positive strand and the negative strand are substantially complementary, and wherein the double stranded nucleic acid molecule has a first end and a second end;
covalently attaching a first sequencing adapter to the positive strand at the first end of the double stranded nucleic acid molecule;
covalently attaching a second sequencing adapter to the negative strand at the first end of the double stranded nucleic acid molecule; and
covalently attaching a first end of a single hairpin adapter to the positive strand at the second end of the double stranded nucleic acid molecule and covalently attaching a second end of the single hairpin adapter to the negative strand at the second end of the double stranded nucleic acid molecule, wherein the single hairpin adapter covalently joins the positive strand and the negative strand into a single covalently linked duplex strand for self-correction of sequencing errors.

2. The method of claim 1, wherein the single hairpin adapter is a partially double stranded nucleic acid molecule that has a secondary structure comprising a double stranded stem domain and a loop domain, wherein the stem domain comprises each end of the hairpin adapter to covalently attach to the positive end and the negative end of the second end of the double stranded nucleic acid molecule.

3. The method of claim 1, wherein the single hairpin adapter comprises a number (N) of nucleotides, wherein each nucleotide is selected independently, and wherein Nis an integer selected from 6 to 300.

4. The method of claim 2, wherein the loop domain comprises a unique molecule identifier (UMID) sequence.

5. The method of claim 4, wherein the loop domain comprises a secondary index sequence adjacent to the UMID sequence.

6. The method of claim 1, further comprising producing the double stranded nucleic acid molecule by (i) shearing a larger double stranded nucleic acid molecule; (ii) enzymatically fragmenting a larger double stranded nucleic acid molecule; or (iii) producing the double stranded nucleic acid molecule by transposon mediated fragmentation.

7. (canceled)

8. The method of claim 6, wherein the double stranded nucleic acid molecule has an overhang end or a blunt end.

9-10. (canceled)

11. The method of claim 1, further comprising adding one or more adenine residues at a 3′ end of the positive strand and/or adding one or more adenine residues at a 3′ end of the negative strand.

12. The method of claim 1, further comprising amplifying the single covalently linked duplex strand to produce a plurality of covalently linked duplex strand amplicons.

13. The method of claim 12, further comprising sequencing at least one covalently linked duplex strand amplicons to produce at least one sequence read comprising a first subsequence corresponding to at least a portion of the positive strand of the double stranded nucleic acid molecule and a second subsequence corresponding to at least a portion of the negative strand of the double stranded nucleic acid molecule.

14. The method of claim 13, wherein only the first subsequence and/or the second subsequence with a unique UMID sequence is analyzed.

15. The method of claim 14, wherein analysis comprises comparing the sequence of the first subsequence to the sequence of the second subsequence and a variation observed in both the first subsequence and the second subsequence is (i) a genetic variation; or (ii) a sequencing error.

16. (canceled)

17. The method of claim 1, wherein the double stranded nucleic acid molecule is a double stranded DNA molecule.

18. The method of claim 1, comprising preparing a plurality of double stranded nucleic acid molecules for sequencing, by performing the method a plurality of times for different double stranded nucleic acid molecules using a plurality of hairpin adapters comprising different UMID sequences.

19. (canceled)

20. A method for detecting one or more genetic variants in a biological sample, the method comprising:
generating a sequencing library by performing the method of claim 1, wherein the sequencing library comprises a plurality of covalently linked duplex strands each comprising a unique UMID sequence;
amplifying at least a portion of the covalently linked duplex strands to produce an amplified sequencing library comprising a plurality of copies of the covalently linked duplex strands;
sequencing at least a portion of the covalently linked duplex strands to obtain at least one sequence read comprising a first subsequence corresponding to at least a portion of the positive strand of the double stranded nucleic acid molecule and a second subsequence corresponding to at least a portion of the negative strand of the double stranded nucleic acid molecule; and
detecting a presence or absence of one or more genetic variants in the biological sample, by comparing the sequence of the first subsequence to the sequence of the second subsequence, wherein one or more variants observed in both subsequences are genetic variants.

21. (canceled)

22. The method of claim 20, wherein a mismatch of one or more variants between the first subsequence and the second subsequence is a sequencing error.

23. A kit comprising:
a first sequencing adapter,
a second sequencing adapter,
a single hairpin adapter,
one or more primers that hybridize to sequences in the first sequencing adapter and/or second sequencing adapter, or a complement thereof, and free nucleotides (dNTPs),
a DNA polymerase,
a ligase, and
written indicia instructing the performance of the method of claim 1.

24. A kit comprising:
a first sequencing adapter,
a second sequencing adapter,
a single hairpin adapter,
a transposome,
one or more primers that hybridize to a transposon sequence,
a DNA polymerase,
a ligase, and
written indicia instructing the performance of the method of claim 1.

25. The kit of claim 23, wherein the single hairpin adapter is a partially double stranded nucleic acid molecule that has a secondary structure comprising a double stranded stem domain and a loop domain, wherein the stem domain comprises each end of the hairpin adapter to covalently attach to the positive end and the negative end of the second end of the double stranded nucleic acid molecule.

26-28. (canceled)

29. The kit of claim 24, wherein the single hairpin adapter is a partially double stranded nucleic acid molecule that has a secondary structure comprising a double stranded stem domain and a loop domain, wherein the stem domain comprises each end of the hairpin adapter to covalently attach to the positive end and the negative end of the second end of the double stranded nucleic acid molecule.
